(19) United States
(12) Patent Application Publication
(10) Pub. No.: US 2026/0122392 A1
(43) Pub. Date: Apr. 30, 2026

(54) CHARGING CASE FOR EARBUDS

(71) Applicants: Reshma N. Kheraj, Oxford, GA (US); NARESH Pulipati, Oxford, GA (US)

(72) Inventors: Reshma N. Kheraj, Oxford, GA (US); NARESH Pulipati, Oxford, GA (US)

(21) Appl. No.: 19/432,908

(22) Filed: Dec. 24, 2025

Related U.S. Application Data
(63) Continuation-in-part of application No. 17/990,495, filed on Nov. 18, 2022, now Pat. No. 12,538,060.
(60) Provisional application No. 62/972,783, filed on Feb. 11, 2020.

(51) Int. Cl. H04R 1/1025; G06F 3/16; H02J 7/70; H04R 1/10

(52) U.S. Cl. CPC H04R 1/1025; G06F 3/162; G06F 3/165; H02J 7/731; H04R 1/1041; H04R 1/1016; H04R 2420/07

(57) ABSTRACT

A charging case for wireless earbuds comprises one or more charging docks configured to receive and charge the earbuds, and ultraviolet (UV) light sources disposed within an interior of the charging case. The UV light sources irradiate the earbuds and an inner volume of the charging case to reduce microbial contamination. A control unit is operably coupled to the charging circuitry, the UV light sources, and one or more sensors, and is configured to control charging and sterilization operations based on one or more conditions, including presence of the earbuds, closure of the charging case, battery status, or user input. The charging case may support prioritization between charging and sterilization, timed sterilization cycles, and safety-based deactivation of the UV light sources. In certain embodiments, the charging case further supports additional functionalities including external device charging, media playback, communication, synchronization with other charging cases, and user status indication.

### CROSS REFERENCE TO RELATED APPLICATIONS

This application is a continuation-in-part of a U.S. patent application Ser. No. 17/990,495 filed on Nov. 18, 2022, which is a continuation-in-part of a Ser. No. 17/223,338 filed on Apr. 6, 2021, which claims priority from a U.S. Provisional Patent Appl. No. 62/972,783, filed on Feb. 11, 2020, all of which are incorporated herein by reference in its entirety.

### FIELD OF INVENTION

The present invention relates generally to a charging case for wireless earbuds, and more particularly, to a multifunctional charging case configured to charge and sterilize wireless earbuds and an interior volume of the charging case.

### BACKGROUND

Wireless earbuds and charging cases for charging the wireless earbuds are known in the art. Such charging cases may be wired or wireless and typically include an inbuilt rechargeable battery for supplying power to the earbuds, wherein the charging case itself is periodically recharged. The charging cases generally include one or more docks configured to securely receive the earbuds and establish electrical contact for charging

Wireless earbuds are frequently handled by users and may, in some instances, be shared between multiple individuals. As a result, the earbuds can become carriers of microorganisms, including pathogenic microorganisms. In particular, soft cushioning portions of the earbuds may readily harbor microorganisms and promote their growth.

Microorganisms may be transferred from a user's hands to the earbuds during handling, and subsequent contact of the earbuds with the ear may increase the risk of ear infections, including fungal infections.

Accordingly, there exists a need for an improved charging case that addresses the foregoing issues. In particular, there is a need for a charging case capable of sterilizing the earbuds to reduce or eliminate microorganisms present on the earbuds and within the charging case.

### SUMMARY OF THE INVENTION

The following provides a simplified summary of one or more embodiments of the present invention to facilitate a basic understanding of its features and advantages. This summary is not an exhaustive overview of all contemplated embodiments and is not intended to identify essential elements or define the full scope of the invention. It merely introduces certain concepts that are described in greater detail in the subsequent sections.

The principal object of the present invention is to provide a charging case for wireless earbuds that are configured to sterilize the earbuds.

Another object of the present invention is to provide a charging case that promotes user health and hygiene.

Still another object of the present invention is to provide a charging case capable of selectively prioritizing between charging and sterilization operations.

Yet another object of the present invention is to provide a charging case that is economical to manufacture.

A further object of the present invention is to provide a charging case that is compact and lightweight.

An additional object of the present invention is to provide a charging case that is portable and convenient for everyday use.

In one aspect, disclosed is a charging case comprising one or more charging docks configured to receive and charge wireless earbuds. The charging case further comprises one or more ultraviolet (UV) light sources disposed within an interior of the charging case and configured to automatically sterilize the earbuds while the earbuds are positioned in the charging docks.

These and other objects and advantages of the embodiments herein will become readily apparent from the following detailed description, taken in conjunction with the accompanying drawings.

### BRIEF DESCRIPTION OF DRAWINGS

The accompanying figures, which are incorporated herein, form part of the specification and illustrate embodiments of the present invention. Together with the description, the figures further explain the principles of the present invention and to enable a person skilled in the relevant arts to make and use the invention.

FIG. 1 illustrates a charging case for wireless earbuds comprising ultraviolet (UV) light-emitting diodes (LEDs), according to an exemplary embodiment of the present invention.

FIG. 2 is a block diagram illustrating an exemplary embodiment of the charging case, according to the present invention.

### DETAILED DESCRIPTION

The subject matter of the present invention will now be described more fully with reference to the accompanying drawings, which form a part of this disclosure and illustrate specific exemplary embodiments. However, it should be understood that the subject matter may be embodied in various forms and is not limited to the specific embodiments set forth herein. Rather, these embodiments are provided by way of example to convey the scope of the invention. It is intended that the claims encompass a broad range of subject matter, including methods, devices, components, and systems. Accordingly, the following detailed description is not intended to be taken in a limiting sense.

As used herein, the term “exemplary” is intended to mean “serving as an example, instance, or illustration.” Any embodiment described as “exemplary” should not be construed as preferred or more advantageous over other embodiments. Similarly, the expression “embodiments of the present invention” does not imply that all embodiments must include all features, advantages, or modes of operation described.

The terminology used herein is for the purpose of describing particular embodiments only and is not intended to be limiting. As used herein, the singular forms “a,” “an,” and “the” include plural references unless the context clearly dictates otherwise. Furthermore, the terms “comprises,” “comprising,” “includes,” and/or “including” specify the presence of stated features, integers, steps, operations, elements, or components, but do not preclude the presence or addition of one or more other features, integers, steps, operations, elements, components, and/or groups thereof.

The following detailed description sets forth the best currently contemplated modes for carrying out exemplary embodiments of the invention. This description is not intended to be limiting, but rather to illustrate the general principles of the invention. The claims of any issued patent will define the scope of the invention.

Disclosed is a charging case for charging wireless earbuds, the charging case comprising one or more ultraviolet (UV) light sources configured to generate ultraviolet radiation for irradiating an inner volume of the charging case. In one exemplary embodiment, the UV light sources comprise UV light-emitting diodes (LEDs) configured to emit radiation in a far UV-C region. The ultraviolet radiation may be within a UV-C wavelength range known to exhibit disinfecting or microbiocidal action. It is to be understood that any radiation suitable for sterilization or disinfection may be employed without departing from the scope of the present invention. The UV LEDs may be activated for a predefined duration that is at least sufficient to expose microorganisms for a time required to reduce a microbial load by at least 99.9 percent.

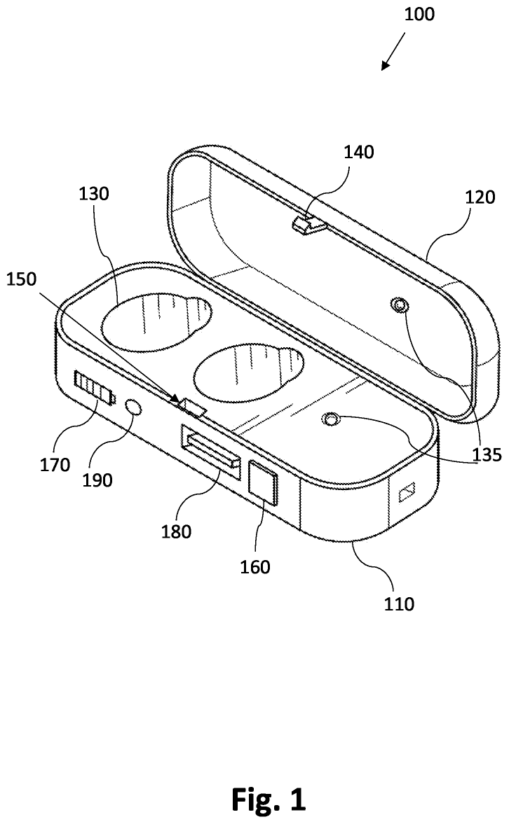

Referring to FIG. 1, illustrated is an exemplary embodiment of a charging case 100 comprising a box body 110 (housing) and a cover 120. The cover 120 is coupled to the box body 110 via a hinge joint. The box body 110 comprises two charging docks 130 configured to receive and support the earbuds for charging. One or more UV LEDs 135 are positioned within the charging case 100 and oriented to irradiate the earbuds when docked. It is to be understood that the number, placement, orientation, and positioning of the earbuds and the UV LEDs may vary based on the size, shape, and configuration of the charging case, without departing from the scope of the present invention.

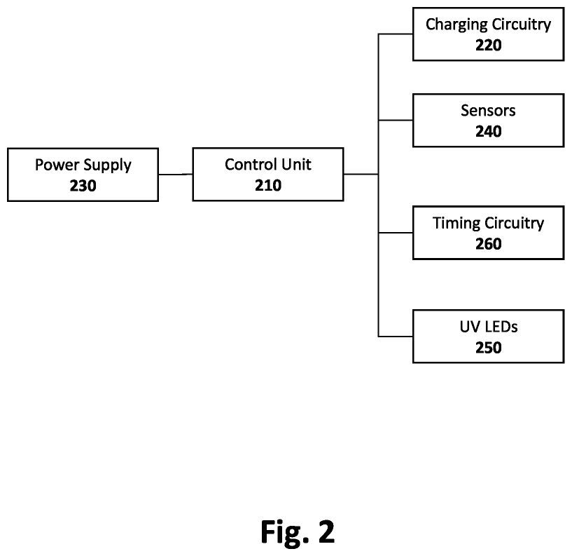

The cover 120 comprises a hook 140 configured to engage a lock member 150 to secure the charging case 100 in a closed state. The charging case 100 may further comprise one or more sensors configured to detect the presence of earbuds in the charging docks 130. Additionally, one or more sensors may be provided to detect opening and closing of the cover 120. As shown in FIG. 2, the charging case 100 further comprises a control unit 210 operably connected to charging circuitry 220, a power supply 230, one or more sensors 240, one or more UV LEDs 250, and timing circuitry 260. The charging circuitry 220 is configured to supply power to the earbuds for charging and may be regulated by the control unit 210. The power supply 230 is configured to supply power to the control unit and other components of the charging case.

The power supply 230 may comprise a rechargeable battery, such as a lithium-ion battery. The battery of the charging case 100 may be charged via a wired connection to an external power source, such as a wall charger. A suitable charging interface, such as a USB port, USB-C port, or any other known input/output interface (not shown), may be provided. In certain embodiments, the same interface may be configured to both charge the battery of the charging case and supply power from the battery to external devices. Alternatively, or additionally, the charging case 100 may be configured for wireless charging. It is to be understood that the charging case may support wired charging, wireless charging, or both. The control unit 210 may regulate distribution of power from the power supply 230 to various components of the charging case.

The charging case 100 comprises one or more UV LEDs 135 configured to irradiate the inner volume of the charging case. In certain embodiments, an inner surface of the charging case may comprise a reflective coating to enhance sterilization efficiency by reflecting UV radiation. The UV radiation is configured to sterilize the earbuds when placed within the charging case 100. The control unit 210 may detect placement of the earbuds in the charging docks 130 via the sensors 240 and may further detect closing of the cover 120, for example by detecting engagement of the hook 140 with the lock member 150, wherein a sensor may be integrated into the lock member 150.

Upon detecting both the presence of the earbuds and closing of the cover, the control unit 210 may automatically supply power to the UV LEDs, thereby irradiating and sterilizing the earbuds and the inner surfaces of the charging case. In certain embodiments, upon detecting closure of the cover without earbuds present, the control unit 210 may activate the UV LEDs to sterilize the inner volume of the empty charging case. Accordingly, when a user removes the earbuds and closes the cover, the charging case may automatically sterilize its interior.

The UV LEDs may be strategically positioned to provide substantially uniform irradiation of the earbuds. In certain embodiments, the charging docks 130 may also include one or more UV LEDs to further enhance sterilization. By way of example, FIG. 1 illustrates one UV LED disposed within the box body and another UV LED disposed on the cover.

The control unit 210 may comprise timing circuitry 260 configured with a predefined duration for which the UV LEDs are activated. Upon expiration of the predefined duration, the control unit 210 may automatically deactivate the UV LEDs. The duration may be preset, modified, or reset by the user or manufacturer. For safety, the control unit 210 may be configured to immediately deactivate the UV LEDs upon detection of opening of the cover while the UV LEDs are active. The control unit 210 may further be configured to manage charging of the earbuds concurrently with or independently from operation of the UV LEDs, such that sterilization may terminate after the predefined duration while charging of the earbuds continues.

In one embodiment, both the UV LEDs and the charging circuitry are powered by a common power supply comprising a rechargeable battery. The battery may be configured with additional capacity to support operation of the UV LEDs. The control unit is configured to manage and balance power delivery between charging of the earbuds and operation of the UV LEDs. For example, when a remaining charge level of the battery falls below a predetermined threshold, the control unit may suspend or limit power supplied to the UV LEDs while continuing to supply power to charge the earbuds. In this manner, the control unit may prioritize charging of the earbuds over sterilization.

In certain embodiments, during initiation of a sterilization cycle, the control unit may allocate a majority or all available power to the UV LEDs to provide an initial burst of ultraviolet radiation sufficient for effective sterilization. The control unit may further provide a user-selectable mode in which sterilization is performed without charging, such as when the earbuds are already sufficiently charged. Additionally, the control unit may automatically determine a charging status of the earbuds and, when the charging status exceeds a predetermined threshold, may selectively activate only the sterilization function. Accordingly, the charging case is capable of dynamically prioritizing between charging and sterilization based on battery status, earbud charge level, user selection, or combinations thereof.

In one exemplary embodiment, the disclosed charging case 100 further comprises a deep-cycle sanitization mode. A dedicated user input element, such as a button 160, may be provided on an exterior surface of the charging case 100. Actuation of the button 160 causes the control unit to activate the UV LEDs for a duration longer than a standard sterilization cycle, thereby performing an extended or deep-cycle sanitization of the earbuds and the inner volume of the charging case.

The disclosed charging case 100 may further comprise one or more charging ports 180 configured to supply power to external electronic devices, such as a mobile phone or other portable electronics. In certain embodiments, the one or more charging ports 180 may be integrated into a wall-charging configuration of the charging case. The charging ports 180 may be powered by the charging circuitry of the disclosed charging case 100 and draw power from the power supply of the charging case.

The charging case 100 may further comprise one or more indicators configured to convey operational status information. A battery status indicator 170 may be configured to display a charge level of the battery and indicate charging of the battery. A UV status indicator 190 may be configured to display an operational status of the UV light sources. For example, the indicator 190 may remain off when the UV light sources are inactive, blink to indicate warming or initialization of the UV light sources, and emit a continuous blue indication to signify normal sterilization operation. In certain embodiments, the indicator 190 may emit a red indication to signify operation in a deep-cycle sanitization mode.

In certain implementations, the disclosed charging case may further incorporate media-player functionality configured to play media content, such as audio tracks or songs. The media content may be transmitted directly to the earbuds, for example via a wireless communication interface. Additionally or alternatively, the charging case may comprise one or more integrated speakers configured to output the media content.

The charging case may further comprise a display screen configured to present operational status information and media information, and one or more user input elements configured to control playback functions, including selection of media content, playlist navigation, and adjustment of sound level. The charging case may be configured to connect to external devices or remote servers through one or more communication networks to access media content. For example, the charging case may connect to a user device, such as a smartphone, and stream media content from the user device to the charging case or directly to the earbuds. In other embodiments, the charging case may connect to one or more media servers, from which media content is transmitted to the charging case.

The charging case may permit a user to configure one or more media servers and to select a media server from among pre-configured media servers. Media content may be organized into playlists created by the user or selected from existing playlists, including default playlists available on the media servers. The charging case may comprise a dedicated control interface for receiving user input and displaying media-related information. Alternatively, or additionally, the charging case may be configured through a connected user device.

Media data may be transmitted to or from the charging case using any suitable data transport mechanism. Media transmission may occur in real time, near real time, or in buffered form. The charging case may receive, transmit, or relay media content using packet-based, stream-based, or message-based communication techniques.

The charging case may further comprise memory configured to buffer media data, wherein a relatively large buffer size may be provided to enable uninterrupted playback. In certain embodiments, the charging case may further comprise memory, whether fixed or removable, configured to store media content locally.

In certain implementations, the disclosed charging case may be configured to connect with one or more other charging cases of the present invention through one or more communication networks. The connected charging cases may synchronize with one another to provide one or more coordinated services. By way of example, a plurality of synchronized charging cases may be configured to play media content in a synchronized manner, wherein one charging case operates as a master device and one or more other charging cases operate as follower or slave devices. It is to be understood that any charging case within the synchronized group may dynamically assume the role of the master device.

The charging case may comprise a user input element, such as a button, configured to initiate synchronization with one or more other charging cases upon actuation. Actuation of the same input element may terminate the synchronization. Synchronization parameters, including device identifiers of charging cases eligible for synchronization, may be preconfigured in each charging case. For example, a group identifier may be defined, and charging cases intended to synchronize may be associated with the group identifier.

Each charging case may be assigned a unique device identifier, which may be linked to one or more group identifiers. Upon detecting another charging case having a matching group identifier, the charging case may automatically establish a communication link with the other charging case. The charging cases may communicate directly with one another via a wireless connection when within network coverage. Additionally or alternatively, the charging cases may communicate via one or more external networks, including the Internet.

In embodiments involving synchronization among a plurality of charging cases, the charging cases may exchange synchronization data using any suitable communication protocol. Synchronization may be achieved using peer-to-peer communication, master-follower coordination, distributed coordination, or combinations thereof, without limitation to any specific networking topology.

Role assignment among synchronized charging cases, including designation of a master or coordinating device, may be static or dynamically determined based on predefined rules, device capability, availability, or user input.

In certain embodiments, the charging case may comprise one or more communication interfaces configured to establish wired or wireless communication with external devices, peer charging cases, user devices, or remote servers. The communication interfaces may operate using any suitable communication protocol, standard, or proprietary signaling scheme, whether now known or later developed.

The communication may be short-range or long-range and may be established directly between devices or indirectly through one or more intermediary devices or networks. The communication interfaces may support device discovery, pairing, authentication, synchronization, data transmission, and control signaling without limitation to any particular protocol or network architecture.

In certain implementations, the charging case may dynamically select or switch between available communication interfaces based on operating conditions, power availability, range, bandwidth, latency, or user preference. Communication may be unidirectional or bidirectional and may occur continuously, periodically, or in response to an event.

In certain embodiments, the disclosed charging case may further support audio and video communication functionality, including voice calls and video conferencing. The charging case may comprise a display configured to present video content during a video communication session. Using the charging case, a user may establish audio and/or video communication with one or more other charging cases or external communication devices.

The charging case may comprise network communication circuitry configured to connect to one or more communication networks, including cellular networks, packet-switched networks, or combinations thereof. The network communication circuitry may support establishment of communication sessions using any suitable communication signaling, transport, or session management techniques, without limitation to a particular protocol.

In certain implementations, multiple synchronized or connected charging cases may participate in a conferencing session, wherein multiple users may actively participate in the session or passively receive audio and/or video content. In further embodiments, the charging case may support anonymous participation in a communication or conferencing session, wherein user identity information is not disclosed to other participants.

In certain embodiments, the charging case may be provided with decorative features, including different colors, surface finishes, and design patterns. In further embodiments, phosphorescent coatings or patterns may be incorporated to enable the charging case to be easily located in low-light or dark environments while also enhancing aesthetic appearance.

In certain embodiments, the charging case and the earbuds may each comprise a locating feature, such as a find button. Actuation of the find button on an earbud may trigger a visual indicator and/or an audible alert on the charging case, and actuation of the find button on the charging case may trigger a visual indicator and/or an audible alert on one or more earbuds. Such locating functionality may assist a user in finding misplaced earbuds or the charging case. The locating functionality may employ any suitable local or remote finding technology, including proximity-based or network-assisted locating techniques.

In certain embodiments, the charging case may comprise one or more microphones configured to capture audio from the surrounding environment and store recorded audio in an internal memory or removable memory. Audio recording may be initiated using a physical input element, software control, or other compatible triggering mechanisms. In certain implementations, the charging case may further support remote listening or two-way audio communication. The charging case may also comprise a camera, thereby enabling use of the charging case for video recording, remote video monitoring, and audio and/or video conferencing.

In certain embodiments, the charging case may comprise network access functionality analogous to that of a mobile communication device, including support for subscriber identity modules or equivalent mechanisms, such that the charging case may be assigned a unique communication identifier. The charging case may thereby be used to make and receive communication sessions independently of a separate user device. A dialing interface, whether physical or virtual, may be provided. In further embodiments, the charging case may be configured to transmit emergency notifications to one or more preconfigured recipients, wherein such notifications may be triggered by a user input, a predefined code, or a voice command. Location information associated with the charging case and/or the earbuds may be made accessible to authorized remote devices.

In certain embodiments, the disclosed charging case may be configured to supply power to external electronic devices, such as smartphones or other portable electronics. The charging case may support multiple charging techniques, including wired, wireless, contact-based, or combinations thereof. One or more charging ports may be provided for wired power delivery, and wireless power transfer functionality may also be supported.

In certain embodiments, the charging case may comprise timing circuitry configured to control durations of various operations. By way of example, timing parameters may be set to limit a duration of media playback, communication sessions, or other operational modes of the charging case.

In certain embodiments, the disclosed charging case may be configured to operate as an electronic digital metronome, generating periodic audio, visual, or haptic signals at a selectable tempo.

In certain embodiments, the disclosed charging case may incorporate digital assistant functionality, enabling execution of voice-activated commands and control of external devices. The charging case may be configured to interact with other electronic devices to perform control operations, such as controlling appliances or other connected equipment.

In certain embodiments, the earbuds may comprise self-diagnostic functionality. Upon detection of a malfunction, damage, or operational error in one or more earbuds, the charging case may generate a notification indicative of the detected condition.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. A charging case for earbuds, comprising:
a housing having a base, a front wall, and a rear wall, the housing defining an inner volume;
a cover coupled to the housing, the cover movable between an open state and a closed state;
one or more charging docks disposed within the inner volume of the housing and configured to receive and electrically charge one or more earbuds;
one or more ultraviolet (UV) light sources disposed within the housing and configured to irradiate at least the one or more earbuds;
a first sensor configured to detect receipt of at least one earbud in a respective charging dock;
a second sensor configured to detect opening and closing of the cover; and
a control unit operably coupled to the one or more UV light sources, the first sensor, the second sensor, and the one or more charging docks, wherein the control unit is configured to:
receive a first signal from the first sensor indicating receipt of the at least one earbud in the respective charging dock;
receive a second signal from a second sensor indicating closure of the cover; and
in response to receiving both the first signal and the second signal, activate the one or more UV light sources for a predetermined duration.

2. The charging case according to claim 1, wherein the control unit is further configured to:
receive a third signal from the first sensor indicating removal of the at least one earbud from the respective charging dock
upon receiving the third signal, receive a fourth signal indicating closing of the cover, and
upon receiving the fourth signal, turn on the one or more ultraviolet (UV) light sources.

3. The charging case according to claim 1, wherein the one or more UV light sources comprise UV light-emitting diodes configured to emit radiation having disinfecting or microbiocidal properties.

4. The charging case according to claim 3, wherein one UV light-emitting diode of the UV light-emitting diodes is positioned in the housing, and one UV light-emitting diode of the UV light-emitting diodes is positioned on an inner surface of the cover.

5. The charging case according to claim 1, wherein the charging case further comprises an integrated rechargeable battery, wherein the control unit and the one or more ultraviolet (UV) light sources are powered by the integrated rechargeable battery.

6. The charging case of claim 1, wherein the control unit is configured to prioritize charging of the one or more earbuds over operation of the one or more UV light sources when a remaining charge level of a power supply falls below a predetermined threshold.

7. The charging case of claim 1, wherein the control unit supports a sterilization-only mode in which the one or more UV light sources are activated without charging the wireless earbuds.

8. The charging case of claim 1, further comprising a user input element configured to initiate a deep-cycle sanitization mode in which the one or more UV light sources are activated for a duration longer than a standard sterilization cycle.

9. The charging case of claim 1, wherein a plurality of charging cases are configured to synchronize operation, and one charging case is designated as a coordinating device.

10. The charging case of claim 9, wherein the operation is playback of music by the coordinating device, and the other charging cases play the same music.

11. The charging case of claim 9, wherein each of the plurality of charging cases has a unique device ID and is associated with a group ID, wherein the group ID is configured in each of the plurality of charging cases, wherein the plurality of charging cases connects with each other based on the group ID.

12. The charging case of claim 1, wherein the control unit is further configured to stream media content to the one or more earbuds.

13. The charging case of claim 12, wherein the control unit is further configured to connect to a media server for receiving the media content.

14. The charging case of claim 12, wherein the charging case further comprises a speaker, a microphone, and a GPS circuitry operably coupled to the control unit.

15. The charging case of claim 14, wherein the charging case further comprises user input elements operably coupled to the control unit and configured to control playback functions.

16. A charging case for earbuds, comprising:
a housing;
a cover coupled to the housing, the cover movable between an open state and a closed state;
one or more charging docks disposed within the housing and configured to receive and electrically charge one or more earbuds;
one or more ultraviolet (UV) light sources disposed within the housing and configured to irradiate at least the one or more earbuds; and
a control unit operably coupled to the one or more ultraviolet (UV) light sources and the one or more charging docks, the control unit is configured to:
determine a current charge status of the one or more earbuds being charged by the one or more charging docks; and
activate the one or more UV light sources when the current charge status exceeds a predefined threshold while the one or more earbuds are not fully charged.

17. The charging case of claim 16, wherein the control unit is further configured to:
while the one or more ultraviolet (UV) light sources are active, suspend charging of the one or more earbuds; and
after expiration of a predefined duration, deactivate the one or more UV light sources and resume charging of the one or more earbuds.

18. A method of operating a charging case for wireless earbuds, the charging case comprising:
a housing having a base, a front wall, and a rear wall, the housing defining an inner volume,
a cover coupled to the housing, the cover movable between an open state and a closed state,
one or more charging docks disposed within the inner volume of the housing and configured to receive and electrically charge one or more earbuds,
one or more ultraviolet (UV) light sources disposed within the housing and configured to irradiate at least the one or more earbuds,
a first sensor configured to detect receipt of at least one earbud in a respective charging dock,
a second sensor configured to detect opening and closing of the cover, and
a control unit operably coupled to the one or more UV light sources, the first sensor, the second sensor, and the one or more charging docks;
the method comprising:
receiving a first signal from the first sensor indicating receipt of the at least one earbud in the respective charging dock;
receiving a second signal from a second sensor indicating closure of the cover; and
in response to receiving both the first signal and the second signal, activate the one or more UV light sources for a predetermined duration.

19. The method according to claim 18, further comprising:
receiving a third signal from the first sensor indicating removal of the at least one earbud from the respective charging dock
upon receiving the third signal, receiving a fourth signal indicating closing of the cover, and
upon receiving the fourth signal, turning on the one or more ultraviolet (UV) light sources.

20. The method according to claim 18, further comprising:
determine a current charge status of the one or more earbuds being charged by the one or more charging docks; and
activating the one or more UV light sources when the current charge status exceeds a predefined threshold while the one or more earbuds are not fully charged.
